(19) United States
(12) Patent Application Publication
(10) Pub. No.: US 2026/0212570 A1
(43) Pub. Date: Jul. 23, 2026

(54) IMAGE RECONSTRUCTION METHOD, IMAGE PROCESSING APPARATUS, AND MAGNETIC RESONANCE IMAGING APPARATUS

(71) Applicant: CANON KABUSHIKI KAISHA, Tokyo (JP)

(72) Inventors: Hideaki KUTSUNA, Kawasaki-shi Kanagawa (JP); Mitsuhiro BEKKU, Utsunomiya Tochigi (JP); Shohei HAMANAGA, Fukuoka Fukuoka (JP)

(73) Assignee: CANON KABUSHIKI KAISHA, Tokyo (JP)

(21) Appl. No.: 19/454,665

(22) Filed: Jan. 21, 2026

(30) Foreign Application Priority Data
Jan. 21, 2025 (JP) 2025-008538

(51) Int. Cl. G06T 12/10; G06T 5/20; G06T 5/50; G16H 30/20; G16H 30/40

(52) U.S. Cl. CPC G06T 12/10; G06T 5/20; G06T 5/50; G16H 30/20; G16H 30/40; G06T 2207/20221

(57) ABSTRACT

An image reconstruction method according to an embodiment includes performing an interpolation estimation process on a first magnetic resonance signal collected through a first collection using a plurality of coils while undersampling and including auto-calibration signals (ACS) to generate image data for each coil based on the ACS, and reconstructing a magnetic resonance image subjected to a sensitivity encoding (SENSE) reconstruction process based on a separately collected sensitivity map obtained through a second collection different from the first collection and on the image data for each coil.

### CROSS-REFERENCE TO RELATED APPLICATIONS

This application is based upon and claims the benefit of priority from Japanese Patent Application No. 2025-008538, filed Jan. 21, 2025, the entire contents of which are incorporated herein by reference.

### FIELD

Embodiments described herein relate generally to an image reconstruction method, an image processing apparatus, and a magnetic resonance imaging apparatus.

### BACKGROUND

As reconstruction methods for magnetic resonance images, there are known methods such as Generalized Autocalibrating Partial Parallel Acquisition (GRAPPA) (U.S. Pat. No. 6,841,998), Autocalibration Reconstruction for Cartesian imaging (ARC) (U.S. Pat. No. 7,692,435), and the like.

As other reconstruction methods, there are known Sensitivity Encoding (SENSE), Compressed Sensing (CS), Machine Learning (ML) reconstruction, and the like. As one method for obtaining a sensitivity map used in SENSE/CS/ML reconstruction, an Extended SPIRiT (ESPIRiT) method (“An Eigenvalue Approach to Autocalibrating Parallel MRI: Where SENSE Meets GRAPPA”, Martin Uecker et. al., Magnetic Resonance in Medicine, 71,990-1001, 2014) is known. In this method, a correlation between coils is obtained as a sensitivity map from auto-calibration signals (ACS).

However, if the field of view (FOV) is insufficient in a phase encoding (PE) direction or a slice encoding (SE) direction, aliasing may occur in the image.

### BRIEF DESCRIPTION OF THE DRAWINGS

FIG. 1 is a diagram illustrating an example of a configuration of a magnetic resonance imaging apparatus according to an embodiment;

FIG. 2 is a flowchart illustrating an example of a flow of processing according to the embodiment;

FIG. 3 is a diagram illustrating magnetic resonance signals acquired by the magnetic resonance imaging apparatus according to the embodiment;

FIG. 4 is a flowchart illustrating an example of processing of step S200 in FIG. 2 in more detail;

FIG. 5 is a flowchart illustrating another example of the processing of step S200 in FIG. 2 in more detail;

FIG. 6 is a diagram illustrating processing according to the embodiment;

FIG. 7 is a diagram illustrating processing according to the embodiment; and

FIG. 8 is a diagram illustrating processing according to the embodiment.

### DETAILED DESCRIPTION

An image reconstruction method according to an embodiment includes performing an interpolation estimation process on a first magnetic resonance signal collected through a first collection using a plurality of coils while undersampling and including auto-calibration signals (ACS) to generate image data for each coil based on the ACS, and reconstructing a magnetic resonance image subjected to a sensitivity encoding (SENSE) reconstruction process based on a separately collected sensitivity map obtained through a second collection different from the first collection and on the image data for each coil.

Various Embodiments will be described hereinafter with reference to the accompanying drawings.

### Embodiment

Hereinafter, embodiments of an image reconstruction method, an image processing apparatus, and a magnetic resonance imaging apparatus will be described in detail with reference to the drawings.

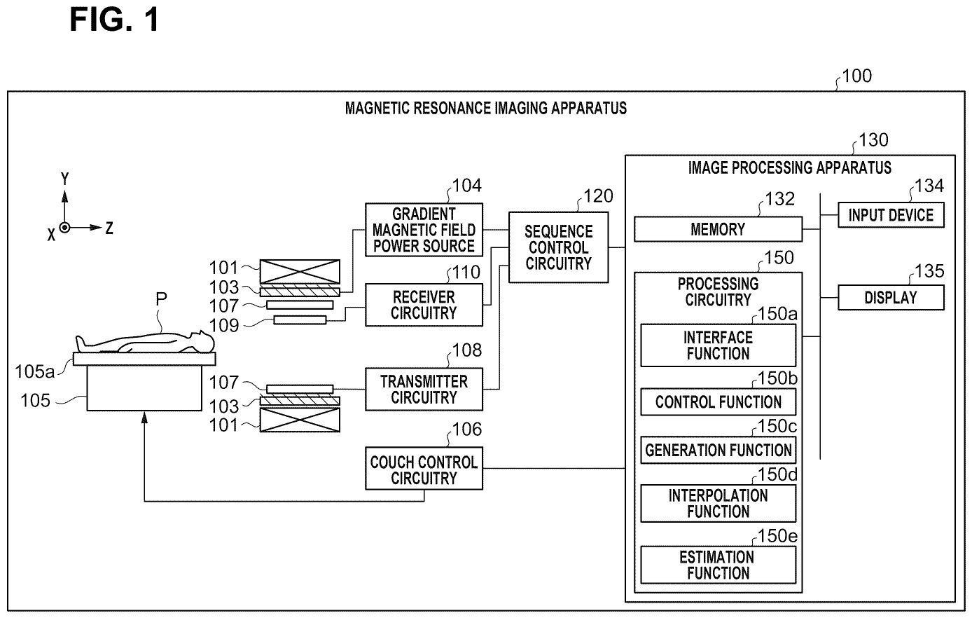

FIG. 1 is a block diagram illustrating a magnetic resonance imaging apparatus 100 according to a first embodiment. As illustrated in FIG. 1, the magnetic resonance imaging apparatus 100 includes a static magnetic field magnet 101, a static magnetic field power source (not illustrated), a gradient magnetic field coil 103, a gradient magnetic field power source 104, a couch 105, couch control circuitry 106, a transmission coil 107, transmitter circuitry 108, a reception coil 109, receiver circuitry 110, sequence control circuitry 120 (sequence control unit), and a computer 130 (also referred to as an “image processing apparatus”). The magnetic resonance imaging apparatus 100 does not include a subject P (for example, a human body). The configuration illustrated in FIG. 1 is merely an example. For example, components in the sequence control circuitry 120 and the computer 130 may be integrated or separated as appropriate.

The static magnetic field magnet 101 is a magnet formed in a hollow, substantially cylindrical shape, and generates a static magnetic field in an internal space. The static magnetic field magnet 101 is, for example, a superconducting magnet or the like. As another example, the static magnetic field magnet 101 may be a permanent magnet.

The gradient magnetic field coil 103 is a coil formed in a hollow, substantially cylindrical shape, and is arranged on an inner side of the static magnetic field magnet 101. The gradient magnetic field coil 103 is formed by a combination of three coils corresponding to X, Y, and Z axes that are orthogonal to each other, and the three coils are individually supplied with electric current from the gradient magnetic field power source 104 to generate gradient magnetic fields in which magnetic field strengths change along the X, Y, and Z axes. The gradient magnetic fields of the X, Y, and Z axes generated by the gradient magnetic field coil 103 are, for example, a slice gradient magnetic field Gs, a phase encoding gradient magnetic field Ge, and a readout gradient magnetic field Gr. The gradient magnetic field power source 104 supplies electric current to the gradient magnetic field coil 103.

The couch 105 includes a couchtop 105a on which the subject P is placed. Under the control of the couch control circuitry 106, the couchtop 105a in a state where the subject P is placed thereon is inserted into a cavity (imaging port) of the gradient magnetic field coil 103. Normally, the couch 105 is disposed such that the longitudinal direction thereof is parallel to the central axis of the static magnetic field magnet 101. Under the control of the computer 130, the couch control circuitry 106 drives the couch 105 to move the couchtop 105a in the longitudinal direction and the up-down direction.

The transmission coil 107 is arranged on an inner side of the gradient magnetic field coil 103, and receives radio frequency (RF) pulses supplied from the transmitter circuitry 108 to generate a high-frequency magnetic field. The transmitter circuitry 108 supplies the transmission coil 107 with RF pulses corresponding to a Larmor frequency determined by the type of target atom and the magnetic field strength.

The reception coil 109 is arranged on an inner side of the gradient magnetic field coil 103, and receives magnetic resonance signals (hereinafter, referred to as “MR signals” as necessary) emitted from the subject P under an influence of the high-frequency magnetic field. Upon receipt of the magnetic resonance signals, the reception coil 109 outputs the received magnetic resonance signals to the receiver circuitry 110.

The above-described transmission coil 107 and reception coil 109 are merely examples. These coils may be configured by combining one or more of a coil having only a transmitting function, a coil having only a receiving function, and a coil having both the transmitting and receiving functions.

The receiver circuitry 110 detects the magnetic resonance signals output from the reception coil 109 and generates magnetic resonance data based on the detected magnetic resonance signals. Specifically, the receiver circuitry 110 generates the magnetic resonance data by digitally converting the magnetic resonance signals output from the reception coil 109. The receiver circuitry 110 also transmits the generated magnetic resonance data to the sequence control circuitry 120. The receiver circuitry 110 may be provided on a gantry device side that includes the static magnetic field magnet 101, the gradient magnetic field coil 103, and the like. Furthermore, the reception coil 109 may be provided with some of the functions of the receiver circuitry 110, such as digital conversion of the magnetic resonance signals.

The sequence control circuitry 120 drives the gradient magnetic field power source 104, the transmitter circuitry 108, and the receiver circuitry 110 based on sequence information transmitted from the computer 130 to perform imaging of the subject P. The sequence information here is information that defines a procedure for performing imaging. The sequence information defines the strength of the electric current supplied by the gradient magnetic field power source 104 to the gradient magnetic field coil 103 and the timing for supplying the electric current, the strength of the RF pulses supplied by the transmitter circuitry 108 to the transmission coil 107 and the timing for applying the RF pulses, the timing for the receiver circuitry 110 to detect the magnetic resonance signal, and the like. For example, the sequence control circuitry 120 is integrated circuitry such as an application specific integrated circuit (ASIC) or a field programmable gate array (FPGA), or electronic circuitry such as a central processing unit (CPU) or a micro processing unit (MPU). The pulse sequence executed by the sequence control circuitry 120 will be described in detail below.

Upon receipt of the magnetic resonance data from the receiver circuitry 110 as a result of driving the gradient magnetic field power source 104, the transmitter circuitry 108, and the receiver circuitry 110 to perform imaging of the subject P, the sequence control circuitry 120 transmits the received magnetic resonance data to the computer 130.

The computer 130 performs overall control of the magnetic resonance imaging apparatus 100 and performs image generation and the like. The computer 130 includes a memory 132, an input device 134, a display 135, and processing circuitry 150. The processing circuitry 150 includes an interface function 150a, a control function 150b, a generation function 150c, and an interpolation function 150d.

In the first embodiment, processing functions performed by the interface function 150a, the control function 150b, the generation function 150c, and the interpolation function 150d are stored in the memory 132 in the form of computer-executable programs. The processing circuitry 150 is a processor that implements the functions corresponding to the programs by reading the programs from the memory 132 and executing the programs. In other words, the processing circuitry 150 having read the programs has the functions illustrated in the processing circuitry 150 in FIG. 1. In FIG. 1, the processing functions to be implemented by the interface function 150a, the control function 150b, the generation function 150c, and the interpolation function 150d are described to be implemented by the single processing circuitry 150. Alternatively, the processing circuitry 150 may be configured with a plurality of independent processors combined, and the individual processors may execute respective programs to implement the functions. In other words, the above-described functions may be configured as programs, and the single processing circuitry 150 may execute the programs. As another example, a specific function may be implemented in dedicated, independent program execution circuitry. In FIG. 1, the interface function 150a, the control function 150b, the generation function 150c, and the interpolation function 150d are examples of a reception unit, a control unit, a generation unit, and an interpolation unit, respectively. The sequence control circuitry 120 is an example of a sequence control unit.

The term “processor” used in the above description refers to circuitry such as a CPU, a graphical processing unit (GPU), an ASIC, or a programmable logic device (for example, a Simple Programmable Logic Device (SPLD), a Complex Programmable Logic Device (CPLD), and a FPGA). The processor implements the functions by reading and executing the programs stored in the memory 132.

Instead of storing the programs in the memory 132, the programs may be directly incorporated in the circuitry of the processor. In this case, the processor implements the functions thereof by reading and executing the programs incorporated in the circuitry. The couch control circuitry 106, the transmitter circuitry 108, the receiver circuitry 110, and the like are configured in a similar manner by electronic circuitry such as the above-described processor.

The processing circuitry 150 uses the interface function 150a to transmit the sequence information to the sequence control circuitry 120, and receive the magnetic resonance data from the sequence control circuitry 120. Upon receipt of the magnetic resonance data, the processing circuitry 150 having the interface function 150a stores the received magnetic resonance data in the memory 132.

The magnetic resonance data stored in the memory 132 is arranged in k-space by the control function 150b. As a result, the memory 132 stores k-space data.

The memory 132 stores the magnetic resonance data received by the processing circuitry 150 having the interface function 150a, the k-space data arranged in k-space by the processing circuitry 150 having the control function 150b, the image data generated by the processing circuitry 150 having the generation function 150c, and the like. For example, the memory 132 is a semiconductor memory element such as a random access memory (RAM) or a flash memory, a hard disk, an optical disk, or the like.

The input device 134 accepts various instructions and information input from an operator. The input device 134 is, for example, a pointing device such as a mouse or a trackball, a selection device such as a mode switching switch, or an input device such as a keyboard. The display 135 displays a graphical user interface (GUI) for accepting input of imaging conditions, images generated by the processing circuitry 150 having the generation function 150c, and the like, under the control of the processing circuitry 150 having the control function 150b. The display 135 is, for example, a display device such as a liquid crystal display.

The processing circuitry 150 uses the control function 150b to perform overall control of the magnetic resonance imaging apparatus 100 to control imaging, image generation, image display, and the like. For example, the processing circuitry 150 having the control function 150b accepts input of imaging conditions (imaging parameters and the like) on the GUI and generates sequence information according to the accepted imaging conditions. The processing circuitry 150 having the control function 150b also transmits the generated sequence information to the sequence control circuitry 120.

The processing circuitry 150 uses the generation function 150c to read k-space data from the memory 132 and generates an image by performing a reconstruction process such as Fourier transformation on the read k-space data.

Next, the background of the embodiment will be described.

As reconstruction methods for magnetic resonance images, there are known methods such as Generalized Autocalibrating Partial Parallel Acquisition (GRAPPA), Autocalibration Reconstruction for Cartesian imaging (ARC), and the like. In these methods, a convolution kernel is obtained from data at the center of k-space, called auto-calibration signals (ACS), and undersampled data is complemented to reconstruct fully sampled data from the undersampled data.

As other reconstruction methods, there are known Sensitivity Encoding (SENSE), Compressed Sensing (CS), Machine Learning (ML) reconstruction, and the like. As one method for obtaining a sensitivity map used in SENSE/CS/ML reconstruction, an Extended SPIRit (ESPIRit) method (“An Eigenvalue Approach to Autocalibrating Parallel MRI: Where SENSE Meets GRAPPA”, Martin Uecker et. al., Magnetic Resonance in Medicine, 71,990-1001, 2014) is known. In this method, a correlation between coils is obtained as a sensitivity map from ACS. The sensitivity map obtained from data collected so as to include the ACS will be referred to as a self-map or self-sensitivity map because a separate scan for acquiring the sensitivity map is not required.

However, if the field of view (FOV) is insufficient in a phase encoding (PE) direction or a slice encoding (SE) direction, aliasing may occur in the image.

Thus, in a magnetic resonance imaging method according to the embodiment, for a reconstruction image for each coil based on ACS, SENSE reconstruction synthesis is performed using a separately collected sensitivity map obtained through an additional scan. The SENSE reconstruction process is also referred to as SENSE unfolding, coil combination, or the like. The separately collected sensitivity map is generated based on signals obtained in a scan other than the main scan including ACS.

More specifically, in the image reconstruction method according to the embodiment, an interpolation estimation process is performed on first magnetic resonance signals collected through a first collection using a plurality of coils while undersampling and including ACS, where the interpolation estimation process generates image data for each coil based on the ACS, and then a magnetic resonance image subjected to the SENSE reconstruction process is reconstructed based on a separately collected sensitivity map obtained through a second collection different from the first collection and on the image data for each coil.

The image processing apparatus according to the embodiment includes an interpolation unit and a reconstruction unit. The interpolation unit performs the interpolation estimation process on the first magnetic resonance signals that are collected through the first collection using a plurality of coils while undersampling and including ACS, and generates the image data for each coil based on the ACS. The reconstruction unit reconstructs the magnetic resonance image subjected to the SENSE reconstruction process based on a separately collected sensitivity map obtained through the second collection different from the first collection and on the interpolation estimation process.

The magnetic resonance imaging apparatus according to the embodiment also includes a sequence control unit that performs the first collection using a plurality of coils while undersampling and including ACS, in addition to the above-described interpolation unit and reconstruction unit.

This enables highly accurate parallel imaging using a self-sensitivity map or self-calibration, performing data interpolation for an undersampled portion while effectively reducing artifacts due to aliasing.

In addition to reducing artifacts due to aliasing, it is also possible to suppress artifacts due to magnetic field distortion (annefact) and artifacts due to air regions (ghost).

In the present embodiment, a reconstruction method capable of generating a self-sensitivity map based on ACS or the like is used. Thus, it may seem unnecessary to collect a separately collected map obtained by an additional scan and perform image reconstruction using the separately collected sensitivity map. However, for example, by configuring the separately collected sensitivity map to have a FOV in the phase encoding direction that is wider than that of the reconstructed image, or by setting the phase encoding direction of the separately collected sensitivity map to differ from that of the phase encoding direction of the reconstructed image, it is possible to achieve both highly accurate parallel imaging and reduction of aliasing artifacts.

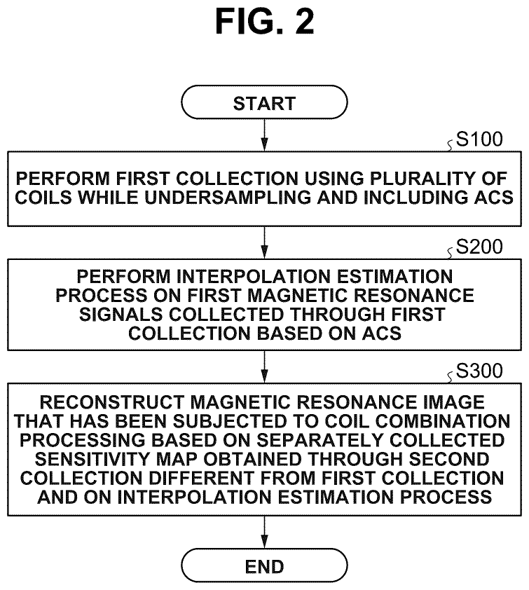

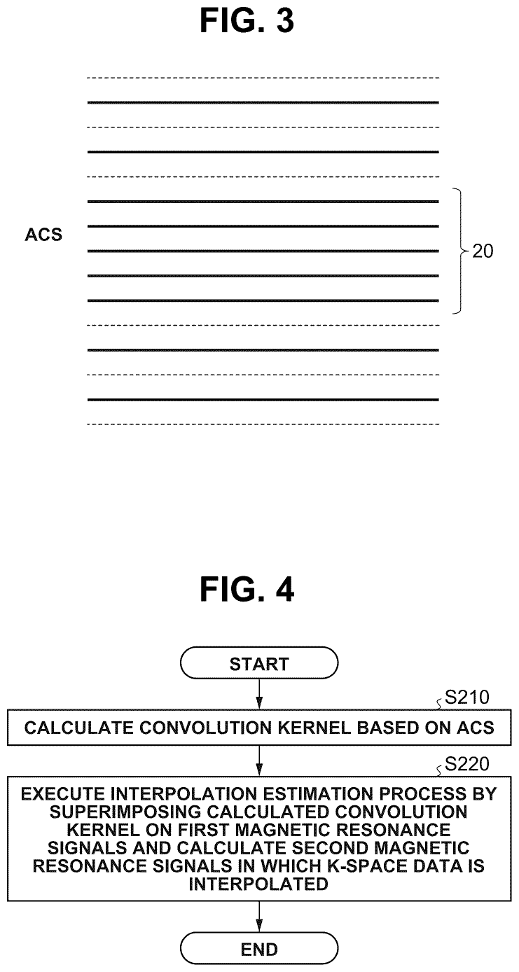

FIG. 2 illustrates a flow of processing of the method according to the embodiment. First, in step S100, the sequence control circuitry 120 performs the first collection using a plurality of coils while undersampling and including ACS, and collects first magnetic resonance signals. FIG. 3 illustrates an example of undersampling. In FIG. 3, solid lines represent k-space where collection is performed, and dotted lines represent k-space where collection is not performed, i.e., undersampled k-space. The sequence control circuitry 120 performs collection while undersampling k-space. In addition, the sequence control circuitry 120 performs the collection including auto-calibration signals 20 near the center of the k-space without undersampling k-space. The sequence control circuitry 120 performs the collection for a plurality of coils. More specifically, the sequence control circuitry 120 performs undersampled collection using a plurality of coils, and collects magnetic resonance data 1 consisting of data from the plurality of coils and including the auto-calibration signals 20.

In step S200, the processing circuitry 150 uses an estimation function 150e to generate image data for each coil based on ACS, for the first magnetic resonance signals collected through the first collection using the plurality of coils while undersampling and including ACS.

FIG. 4 illustrates a flow of processing in step S200 in the case of using, for example, GRAPPA as a reconstruction method. More specifically, steps S210 and S220 in FIG. 4 correspond to step S200 in FIG. 2. In the case of using the reconstruction method such as GRAPPA or ARC in step S200, for example, first, in step S210, the processing circuitry 150 uses the estimation function 150e to calculate a convolution kernel from data on ACS for first magnetic resonance signals collected through the first collection using the plurality of coils and obtained by a multi-coil fast imaging technique. Next, in step S220, the processing circuitry 150 uses the estimation function 150e to execute an interpolation estimation process by superimposing the calculated convolution kernel on the first magnetic resonance signals, and calculates second magnetic resonance signals, which are image data in which k-space data is interpolated.

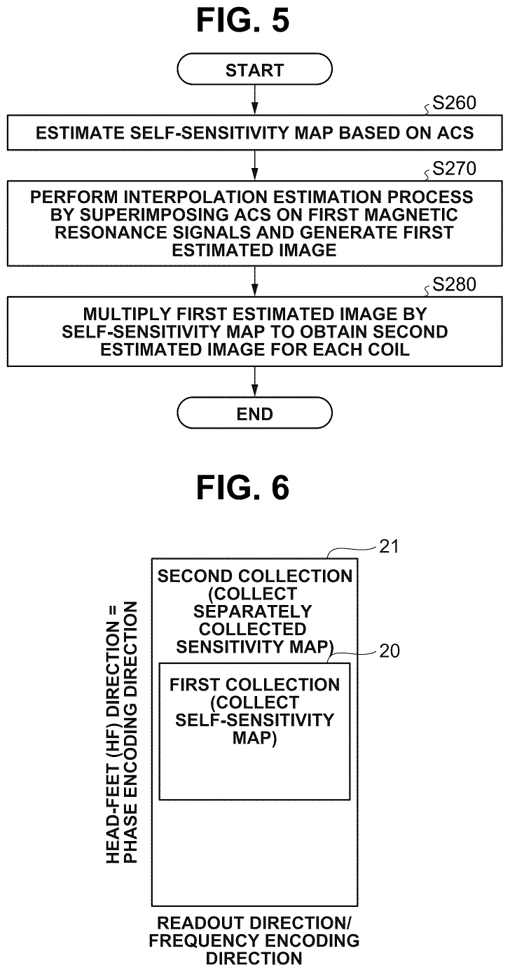

As another example, image reconstruction may be performed using the ESPIRiT method (see “An Eigenvalue Approach to Autocalibrating Parallel MRI: Where SENSE Meets GRAPPA”, Martin Uecker et al., Magnetic Resonance in Medicine, 71,990-1001,2014). FIG. 5 illustrates a flow of processing in step S200 in the case of using the ESPIRiT method, for example. More specifically, steps S260 to S280 in FIG. 5 correspond to step S200 in FIG. 2. In step S260, the processing circuitry 150 uses the estimation function 150e to estimate a self-sensitivity map based on ACS. In step S270, the processing circuitry 150 uses the estimation function 150e to perform an interpolation estimation process by superimposing the ACS on the first magnetic resonance signals, and generates a first estimated image.

Next, in step S280, the processing circuitry 150 uses the estimation function 150e to multiply the first estimated image by the self-sensitivity map to obtain a second estimated image, which is image data for each coil. More specifically, the processing circuitry 150 uses the estimation function 150e to multiply the first estimated image, which is obtained based on ACS, by the self-sensitivity map to generate image data for each coil. In this manner, the processing circuitry 150 uses the estimation function 150e to perform the interpolation estimation process including estimation of the self-sensitivity map based on ACS.

With reference to FIG. 2, in step S300, the processing circuitry 150 uses the generation function 150c to reconstruct a magnetic resonance image that has been subjected to the SENSE reconstruction process, based on the separately collected sensitivity map obtained through the second collection different from the first collection and on the interpolation estimation process performed in step S200. More specifically, the processing circuitry 150 uses the generation function 150c to reconstruct a magnetic resonance image that has been subjected to the SENSE reconstruction process based on the separately collected sensitivity map obtained through the second collection different from the first collection and on the image data for each coil obtained in step S200.

As described above, in step S200, since the reconstruction method capable of generating a self-sensitivity map based on ACS or the like is used, it is possible to perform image reconstruction based on the self-sensitivity map obtained based on the ACS without collecting a separately collected sensitivity map obtained by an additional scan. However, in step S300, by making an aliasing direction of the separately collected sensitivity map obtained through the second collection different from the first collection different from an aliasing direction of the self-sensitivity map, it is possible to, for example, improve data quality in a direction where the data quality is low in the self-sensitivity map, reduce artifacts due to aliasing, and perform highly accurate parallel imaging while shortening the imaging time.

FIG. 6 illustrates a first example of collection of a separately collected sensitivity map. In the first example, a second collection 21 has a wider collection range than a first collection 20 in at least one collection axis direction. As an example, when a Head-Feet (HF) direction is set as the phase encoding direction, the second collection 21 has a wider collection range than the first collection 20 in the phase encoding direction. Accordingly, the separately collected sensitivity map can have a FOV in the phase encoding direction that is wider than that of the reconstructed image.

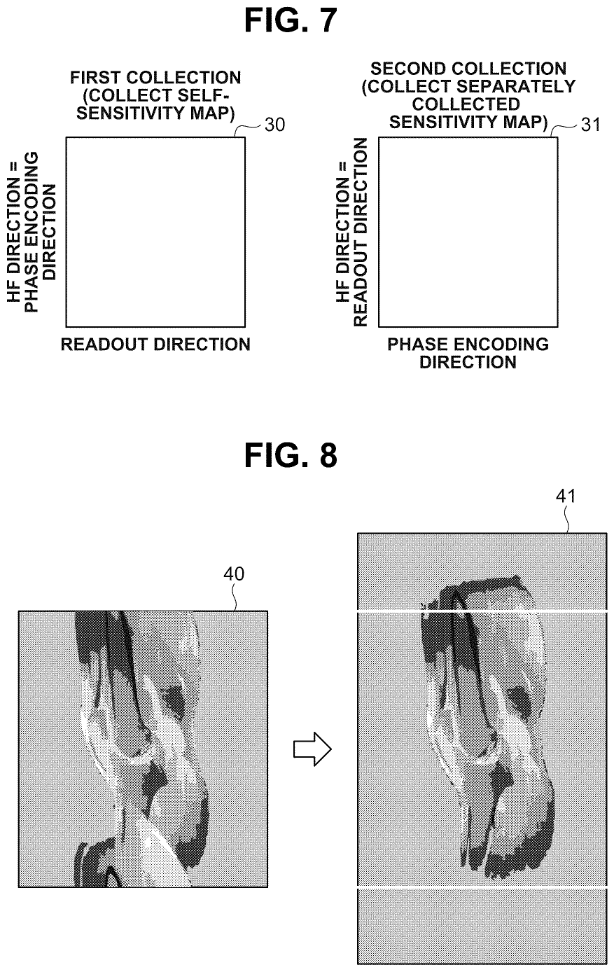

FIG. 7 illustrates a second example of collection of a separately collected sensitivity map. In the second example, a second collection 31 has a phase encoding direction different from that of the first collection 30. As an example, in the self-sensitivity map, the HF direction is the phase encoding direction, whereas in the separately collected sensitivity map, the HF direction is a readout direction. Accordingly, it is possible to reinforce data in a direction in which the sensitivity of the self-sensitivity map is low, thereby improving image quality.

As another example of collection of a separately collected sensitivity map, instead of the second collection 31 in FIG. 7, a two-dimensional (2D) sensitivity map may be collected in which the HF direction is an excitation slice direction.

As described above, in the image reconstruction method according to the embodiment, on the first magnetic resonance signals that are collected through the first collection using a plurality of coils while undersampling and including ACS, the interpolation estimation process that generates image data for each coil based on the ACS is performed, and a magnetic resonance image subjected to the SENSE reconstruction process is reconstructed based on a separately collected sensitivity map obtained by the second collection different from the first collection and on the image data for each coil. Accordingly, it is possible to reduce artifacts due to aliasing, artifacts due to magnetic distortion or air regions, and the like, and perform highly accurate parallel imaging while shortening the imaging time.

The embodiment is not limited to the above. As a modified example of the embodiment, in a case where the separately collected sensitivity map has a sufficiently wide coverage, the processing circuitry 150 may use the generation function 150c to perform a SENSE reconstruction process involving a SENSE unfolding process of two times or more, and for example, generate an image 41 subjected to the SENSE unfolding process of two times or more from an image 40 before unfolding, as illustrated in FIG. 8. Accordingly, it is possible to reduce the contribution of coils that have sensitivity in other regions and improve image quality.

According to at least one of the embodiments described above, it is possible to improve image quality.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An image reconstruction method comprising:
performing an interpolation estimation process on a first magnetic resonance signal collected through a first collection using a plurality of coils while undersampling and including auto-calibration signals (ACS) to generate image data for each coil based on the ACS; and
reconstructing a magnetic resonance image subjected to a sensitivity encoding (SENSE) reconstruction process based on a separately collected sensitivity map obtained through a second collection different from the first collection and on the image data for each coil.

2. The image reconstruction method according to claim 1, further comprising interpolating missing parts due to undersampling by superimposing a convolution kernel calculated based on the ACS on the first magnetic resonance signal.

3. The image reconstruction method according to claim 1, further comprising estimating a self-sensitivity map based on the ACS.

4. The image reconstruction method according to claim 3, further comprising generating the image data for each coil by multiplying a first estimated image obtained based on the ACS by the self-sensitivity map.

5. The image reconstruction method according to claim 1, further comprising performing the SENSE reconstruction process involving a SENSE unfolding process of two times or more.

6. The image reconstruction method according to claim 1, wherein the second collection has a wider collection range than the first collection in at least one collection axis direction.

7. The image reconstruction method according to claim 1, wherein the second collection is different from the first collection in phase encoding direction.

8. An image processing apparatus comprising processing circuitry configured to:
perform an interpolation estimation process on a first magnetic resonance signal collected through a first collection using a plurality of coils while undersampling and including auto-calibration signals (ACS)to generate image data for each coil based on the ACS; and
reconstruct a magnetic resonance image subjected to a sensitivity encoding (SENSE) reconstruction process based on a separately collected sensitivity map obtained through a second collection different from the first collection and on the image data for each coil.

9. A magnetic resonance imaging apparatus comprising processing circuitry configured to:
perform first collection using a plurality of coils while undersampling and including auto-calibration signals (ACS);
perform an interpolation estimation process on a first magnetic resonance signal collected through the first collection to generate image data for each coil based on the ACS; and
reconstruct a magnetic resonance image subjected to a sensitivity encoding (SENSE) reconstruction process based on a separately collected sensitivity map obtained through a second collection different from the first collection and on the image data for each coil.
